(19) United States
(12) Patent Application Publication
(10) Pub. No.: US 2006/0089614 A1
(43) Pub. Date: Apr. 27, 2006

(54) Pant-like absorbent garment having a form-fitted back section

(76) Inventor: Faye A. Bonnin, Appleton, WI (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 10/972,036

(22) Filed: Oct. 22, 2004

(51) Int. Cl. A61F 13/15

(52) U.S. Cl. 604/385.27; 604/385.22

(57) ABSTRACT

A pant-like absorbent garment having a rear body defining member forming a contoured and form-fitted back section. The absorbent garment includes a front region, a back region, and a crotch region with waist and leg openings when fitted about the lower torso of the body. The absorbent garment has an outer garment-side layer or back sheet, a body-side liner or topsheet, and an attached retention portion disposed between a liquid impermeable back sheet and a liquid permeable body-side topsheet. A rear body defining member is attached through the outer layers of the absorbent garment along the longitudinal center line of the back region of the garment, and extends from the crotch area towards the back waist area. In a preferred embodiment the defining member is elasticized. The defining member contracts the layers of the absorbent garment to which it is attached and thereby defines and forms divided and contoured rear buttock sections in the absorbent garment, providing an absorbent garment having a more natural contoured and shaped look when worn by a user.

### FIELD OF THE INVENTION

This invention is directed to personal care absorbent garments, and in particular to pant-like absorbent garments having a rear section that is form-fitted to achieve a contoured natural look, particularly for adult wearers.

### BACKGROUND OF THE INVENTION

Absorbent garments, and in particular pant-like disposable incontinence garments, typically include an absorbent retention portion sandwiched between a backsheet and a topsheet or liner which form the garment. Alternately, such absorbent garments may include an absorbent retention assembly connected to one or more garment body panels. Examples of such pant-like absorbent incontinence garments for adults as well as for children are widely available from various manufacturers. Examples are shown and described in, e.g., U.S. Pat. Nos. 6,733,484, 6,716,205, 6,755,808, and 6,761,711. Typically the absorbent retention portions are secured to the garment backsheet or body panels, including along peripheral portions or side edges of the absorbent retention portion. The leg openings and waist band of such absorbent garments are typically elasticized.

However, the resulting absorbent garment does not conform to the natural shape of the wearer's buttocks and instead presents a flattened and drooping “diaper” look from the rear, as shown in FIGS. 1a and 1b. This “diaper” look can be very apparent when an adult wearer of the absorbent garment wears slacks or other form-fitting clothing. This is an undesirable look for an adult and may negatively affect the wearer's personal dignity and/or the person's desire to appear in public wearing such absorbent garments.

Accordingly, there is a need to provide an adult absorbent garment having a more fitted natural look that better conforms to the natural contours of the wearer's buttocks and is less noticeable. Such a fitted or shaped absorbent garment would enhance the aesthetics of such garments for adults who have the need to wear such garments, particularly for active adults.

### SUMMARY OF THE INVENTION

The present invention is directed to a pant-like absorbent garment, such as an adult incontinence garment, having a rear region that is contoured about the two separate portions of the wearer's buttocks, and thereby presents a more fitted and natural appearance for the wearer of the garment. In a preferred embodiment, an elasticized shaping member is attached to the rear back region of the garment longitudinally along the center of the rear back region of the garment. The shaping member is preferably attached through the absorbent retention portion and the outer layers of the garment. The attached shaping member extends from the crotch area longitudinally in the direction upward towards the rear waist area. The shaping member longitudinally contracts and draws up the center portion of the rear back region of the garment to which it is attached. This shaping member thereby causes formation of two defined and somewhat rounded rear lateral portions of the garment, resulting in an absorbent garment presenting a more natural appearance for the wearer.

The present invention and its objects and advantages will be best understood by reference to the following detailed description taken in conjunction with the accompanying drawings.

### BRIEF DESCRIPTION OF THE DRAWINGS

FIGS. 1a and 1b are rear perspective views of prior art absorbent garments showing the undesirable “diaper” look of the prior art garments.

FIG. 2 is a rear perspective view of the absorbent garment of this invention showing the improved form-fitting natural look.

FIG. 3 is a plan view of a first embodiment of the absorbent garment in an unfastened, stretched and laid-flat condition showing the outer garment-side surface.

FIG. 4 is a partially cut-away plan view of the absorbent garment in an unfastened, stretched and laid-flat condition showing the body-side surface.

FIG. 5 is a plan view of an absorbent garment of this invention in an unfastened, stretched and laid-flat condition showing the body-side surface and having longitudinal containment flaps.

FIG. 6 is a partially cut-away plan view of an alternate embodiment of the absorbent garment in an unfastened, stretched and laid-flat condition showing the body-side surface and having a separate absorbent retention composite attached to one or more garment body sections.

### DETAILED DESCRIPTION OF THE PRESENTLY PREFERRED EMBODIMENTS

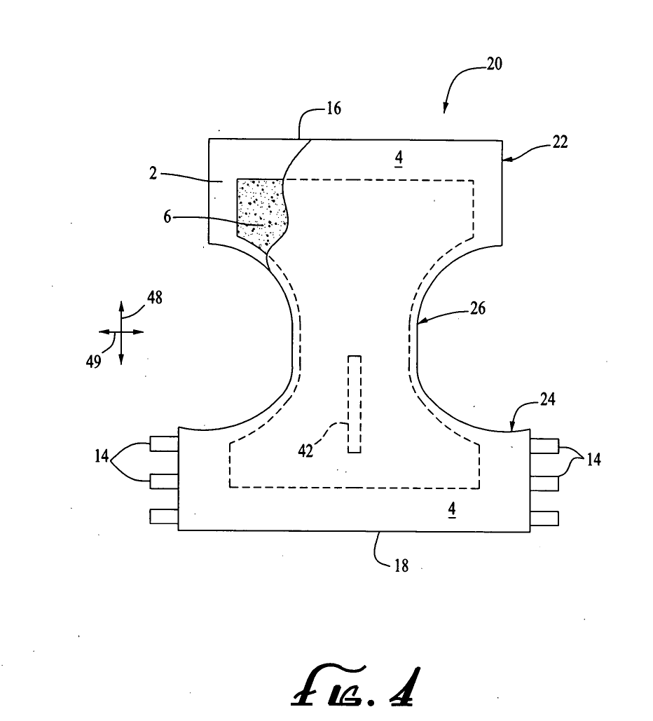

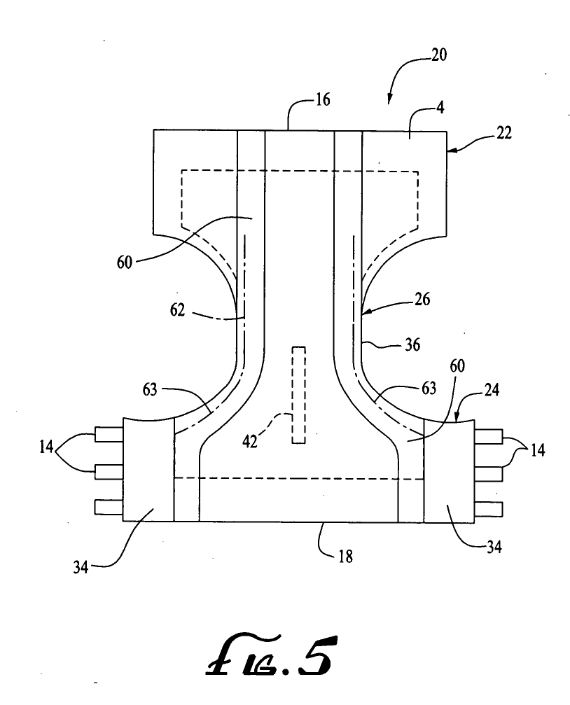

Referring to FIG. 3, a preferred embodiment of the absorbent garment 20 of this invention is shown in an unfastened, stretched and laid-flat state, showing the outer or “garment-side” surface, which is the side facing away from the body of the user and towards the outer garments worn by the user. FIG. 4 shows the “body-side” surface of absorbent garment 20, which is the side closest to the body of the user. FIG. 2 shows absorbent garment 20 in a condition fastened about the body of the wearer to form a pant-like absorbent garment.

As shown in FIGS. 3 and 4, absorbent garment 20 includes a backsheet 2, a topsheet 4, and a retention portion 6 disposed or sandwiched therebetween. The backsheet 2 is preferably liquid impermeable, but may be liquid permeable, e.g., when a barrier layer is used with the retention portion 6. The backsheet 2 prevents bodily fluids and exudates from wetting or otherwise contaminating various bedding or outer garments worn by the user over the absorbent garment. Backsheet 2 can be made of a variety of materials which are known to those skilled in the art. For example, the backsheet can be made from a thin plastic film or other flexible, substantially liquid-impermeable material, such as a polyethylene film. Alternately, it can comprise a woven or nonwoven fibrous web layer which is treated or constructed, partially or wholly, to impart the desired levels of liquid impermeability to selected regions that are adjacent to or proximate the absorbent retention portion. Backsheet 2 may include a gas-permeable nonwoven fabric layer laminated to a polymer film layer which may or may not be gas-permeable. The backsheet typically provides the outercover of the absorbent garment 20. Optionally, the garment may include a separate outercover component (not shown) which is additional to the backsheet.

The topsheet 4 of absorbent garment 20 is shown in FIG. 4, and presents a body-facing surface that is compliant, soft-feeling, and non-irritating to the wearer's skin. Topsheet 4 is sufficiently porous to be liquid permeable, permitting liquid to readily penetrate through its thickness to reach the retention portion 6. Topsheet 4 is also typically employed to help isolate the wearer's skin from liquids. A suitable topsheet can be made from a wide variety of nonwoven and woven materials as will be apparent to those of skill in the art, such as porous foams, reticulated foams, apertured plastic films, natural fibers such as wood or cotton fiber, synthetic fibers such as polyester or polypropylene, or a combination of natural and synthetic fibers. Topsheet 4 may be composed of a substantially hydrophobic material which may optionally be treated with a surfactant or otherwise processed to impart a desired level of wettability and hydrophilicity. As one example, topsheet 4 may be made of a nonwoven polypropylene fabric surface treated with an operative amount of surfactant.

The retention portion 6 of absorbent garment 20 is disposed between backsheet 2 and topsheet 4. Retention portion 6 is indicated entirely by broken lines in FIG. 3, and is shown in the partially cut-away view of FIG. 4. Retention portion 6 extends from the front region 22 of the garment through the crotch region 26 to the back or rear region 24 of the garment. In a preferred embodiment retention portion 6 does not extend the full longitudinal length of absorbent garment 20. The retention portion 6 can be any wettable, hydrophilic absorbent material or combination of materials that preferably tend to swell or expand as they absorb liquid excreted or exuded by the wearer of the garment. Appropriate such materials will be readily apparent to those of skill in the art. For example, the absorbent material of retention portion 6 can be made of airformed, airlaid and/or wetlaid composites of fibers and high absorbency materials, referred to as superabsorbents including superabsorbents made of absorbent gelling material polymers such as polyacrylic acids, and various binding agents used to stabilize the structure. In addition, various foams, absorbent films, and absorbent and superabsorbent fabrics can be used as absorbent materials. Retention portion 6 could also include multiple additional absorbent portions and layers, including surge layers and liquid impermeable barrier layers to enhance the ability of retention portion 6 to absorb and retain liquids and exudates.

Referring to FIGS. 3 and 4, the backsheet 2 and topsheet 4 preferably extend longitudinally and laterally and define the length and width of the absorbent garment 20. The term “longitudinal” as used herein means of or relating to length or the lengthwise direction, and in particular, the direction running between the front and back of the wearer or user of the absorbent garment. The term “laterally” means situated on, directed toward or running from side to side, and in particular, a direction running from the left to the right side of a user of the absorbent garment. For reference, arrows 48 and 49 illustrated in FIGS. 3 and 4 depict the orientation of the longitudinal axis and the lateral axis, respectively, of absorbent garment 20.

In the embodiment shown in FIGS. 3 and 4, the combination of the backsheet 2 and topsheet 4, with the retention portion 6 disposed therebetween, makes up substantially the entire garment and defines the shape or plan surface area of absorbent garment 20. Absorbent garment 20 has a pair of longitudinally opposed Waist edges, which are designated front waist edge 16 and back waist edge 18. When fitted to the wearer's body, the absorbent garment defines a pant-like garment having a front region 22 contiguous with the front waist edge 16, a back region 24 contiguous with back waist edge 18, and a crotch region 26 interconnecting front region 22 and back region 24. When fitted to the wearer's body, the topsheet 4 contacts the wearer and the backsheet 2 contacts the wearer's clothing.

Each of backsheet 2 and topsheet 4 has side flap portions extending laterally outward from the center of the front and back regions forming the sides of the front and back regions. The front side flap portions 32 and the rear side flap portions 34 of backsheet 2 are shown in FIG. 3. Each of backsheet 2 and topsheet 4 has peripheral side edges 36 extending along the two lateral sides of crotch region 26 and extending between the front and rear side flap portions. These peripheral side edges 36 define the leg opening when the absorbent garment 20 is assembled and worn by the user. In a preferred embodiment the peripheral side edges 36 are concave shaped, as shown in FIG. 3.

To further enhance containment of body exudates, the peripheral side edges 36 preferably have elasticized stitching or other elastic members (not shown) secured along the edges in order to form a gasket with the legs of the user. Elasticized stitching or other elastic members can also be secured at the front waist edge 16 and the back waist edge 18 of the absorbent garment. Such elastic members can be operatively joined to backsheet 2 and/or to topsheet 4 along the waist edges and the peripheral side edges, and can extend along part or all of those edges. The elastic members can be formed of any suitable elastic material, as will be known to those skilled in the art, such as strands of natural rubber, synthetic rubber, or thermoplastic elastomeric polymers. The elastic materials can be stretched and adhered to the backsheet or topsheet material, adhered to gathered material, or adhered to the material and then elasticized or shrunk, for example, with the application of heat. As one example, the elastic members may comprise a plurality of dry-spun coalesced multifilament spandex elastomeric threads of Lycra® material.

One or more fastening tabs 14 are attached and extend laterally from each of opposite ends of the rear or front side flap portions, and preferably from the rear side flap portions 34, of the absorbent garment. Fastening tabs 14 can be any of a variety of fastening devices, as will be apparent to those of skill in the art, including tapes having adhesive or other bonding agents applied to one surface, Velcro® fastening systems, buttons, snaps, ties, or other known fastening devices. When the absorbent garment 20 is secured to the user, the fastening tabs 14 are secured to the side flap portions of the other end of the garment. When secured in this way, openings are formed on each side of the garment along the peripheral edges 36 and are shaped to receive the legs of the user. Alternately, front and rear side flap portions 32 and 34 can be secured together to form side seams, wherein the garment is configured as a pant that is slipped on and off the body of the user.

As shown in FIG. 3, a rear body defining member 42 is attached along the longitudinal center line of back region 24 and extends longitudinally from initial point 45 in the crotch area 26 toward the back waist area. Preferably defining member 42 does not extend all the way to the back waist edge 18, but instead has its terminal point 47 about four inches below the back waist edge. In a particularly preferred embodiment the defining member 42 comprises an elastic member or stitching 44 which is stitched through backsheet 2 and through most or all of the thickness of retention portion 6. The defining member 42 may also be attached through topsheet 4 if desired. Where defining member 42 is elasticized, the elastic must be strong enough to contract and “pucker” the layers of the absorbent garment 20 to which it is attached to thereby define and form divided and somewhat rounded buttock portions in the rear region of the garment. This results in an absorbent garment having the more shaped and contoured natural look shown in FIG. 2.

Defining member 42 may alternately comprise other means to separate and shape the two lateral side portions of the back region 24 of absorbent garment 20 to achieve the desired contour about the buttocks of the wearer. For example, a string or cord could be attached along or run through some or all of the layers of the rear portion of the absorbent garment longitudinally along the center of the back region 24, such as between points 45 and 47, and the cord drawn up or cinched to contract and pucker the layers of the absorbent garment to obtain the desired contoured and form-fitted result.

Absorbent garments often contain additional members known as containments flaps which provide a barrier to transverse flow of body exudates. Such containment flaps typically comprise a pair of long, narrow and generally parallel flaps attached along one side of the flaps to the body-side surface of the peripheral sides of the crotch region of the absorbent garment. The containment flaps may also be formed by folded-over portions of the topsheet. The containment flaps typically extend longitudinally towards the front and back waist edges of the absorbent garment. The containment flaps have an unattached edge opposite and generally parallel to the attached edge. Examples of such containment flaps are shown, e.g., in U.S. Pat. Nos. 6,761,711 and 6,716,205. Typically such containment flaps have elastic members along their unattached edges which cause the flaps to assume an upright, generally perpendicular configuration in at least the crotch region, thereby forming containment barriers with the wearer's body when the absorbent garment is worn. Where an absorbent garment of this invention includes containment flaps, additional modifications may be required to obtain the desired fitted look shown in FIG. 2.

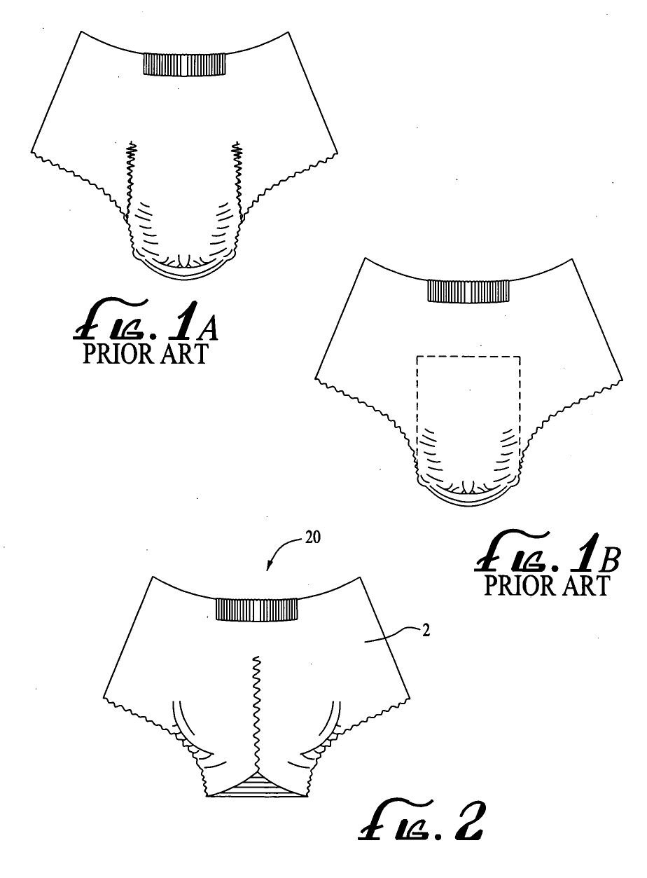

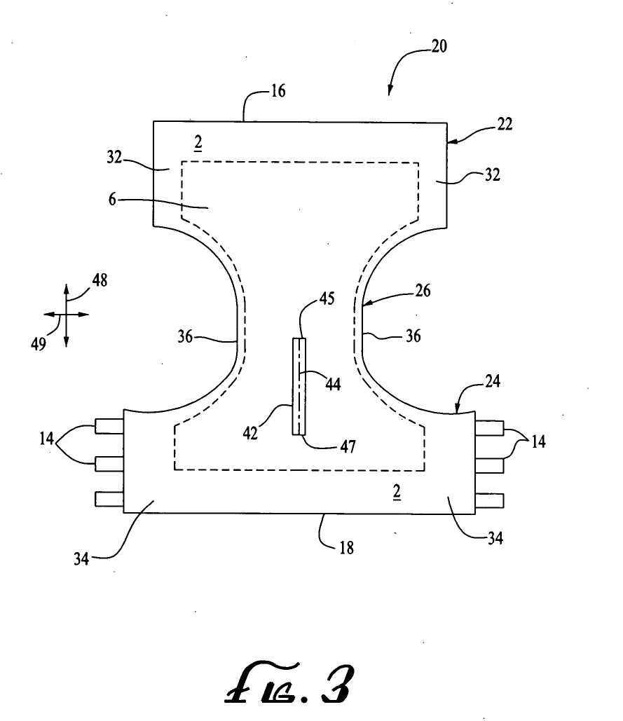

For example, the containment flaps typically have elastic members along some or all of the region where the flaps are attached to the body-side surface of the absorbent garment, including in the crotch region along the peripheral side edges and extending longitudinally towards the front and rear waist edges. Where these containment flaps have elastic members which are attached through the layers of the absorbent garment, such as, e.g., through the backsheet 2 and the retention portion 6 of the garment, the undesirable “diaper” look shown in FIG. 1a will result if this containment flap attachment extends into the rear or back region 24 of the garment.

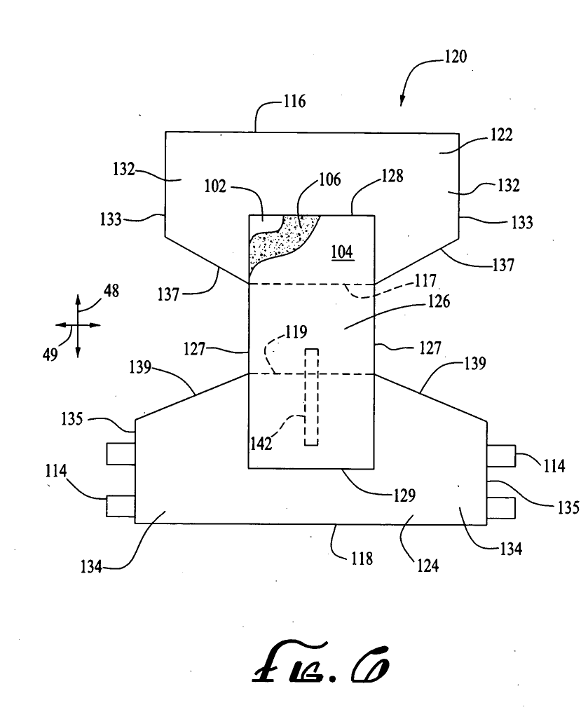

Accordingly, where such containment flaps are included as part of the absorbent garment of this invention, the attachment of the containment flap elastic members to the garment must not extend through the backsheet 2 and retention portion 6 layers of the central portion of the back region 24 of the garment. Instead, as shown in FIG. 5, the longitudinally extending elastic members of the containment flaps must be attached in the back region 24 to the peripheral edges forming the leg openings of the garment. FIG. 5 shows the body-side surface of an absorbent garment 20 of this invention having two attached containment flaps 60 extending from the crotch region 26 along the peripheral side edges 36. The containment flaps 60 are attached to the body-side surface of absorbent garment 20 extending to the front waist edge 16 and the back waist edge 18. The containment flaps 60 have elastic members 62 attached through the layers of the garment along the peripheral side edges 36 of the crotch region, and this attachment extends in a generally straight line into the front region 22 of the garment towards the front waist edge 16. However, in the back or rear region 24 of the garment, the attachment of the containment flap elastic members through the layers of the garment must be made along the peripheral side edges forming the leg openings, such as shown in FIG. 5. FIG. 5 shows the attachment of elastic members 63 of the containment flaps 60 in the back region 24 to be along the peripheral side edges forming the leg openings of the garment extending towards rear side flap portions 34.

Accordingly, in an absorbent garment having containment flaps 60, attachment of the rear body defining member 42 described above, in combination with the positioning of the attachment of the containment flap rear elastic members 63 along the peripheral side edges of back region 24 to form elasticized leg openings, will result in the desired form-fitted and contoured look of the absorbent garment 20, as shown in FIG. 2.

FIG. 6 shows an alternate embodiment of an absorbent garment 120 of this invention which includes a first front body panel 122 and a second rear body panel 124, both of which are connected to an absorbent composite section 126 which forms the crotch region of the garment. Each of the first and second body panels has first and second longitudinally opposed waist and terminal end edges 116, 117, 118, 119. Terminal end edges 117 and 119 of the body panels may be attached to absorbent composite section 126, and they may alternately be attached to each other or to a third crotch body panel (not shown), as desired. Each body panel also has front and back outer side edges 133 and 135 formed along the outer periphery of laterally opposed front and back side flap portions 132 and 134.

The first and second body panels 122 and 124 each have peripheral leg edges 137 and 139, which may be tapered as shown in FIG. 6 or which may extend laterally and without taper from terminal end edges 117 and 119 to outer side edges 133 and 135. Elasticized stitching or other elastic members (not shown) may be secured along waist edges 116 and 118 of the front and back body panels, and may also be secured along peripheral leg edges 137 and 139 and, if desired, along the peripheral edges 127 of absorbent composite 126.

The first and second body panels 122 and 124 each have an outer, garment-side surface and an inner, body-side surface. These body panels may be made of any appropriate material such as those discussed above for the backsheet and the topsheet of the absorbent garment of the first embodiment.

The absorbent composite 126 has first and second longitudinally opposed front and back terminal end regions 128 and 129, and laterally opposed peripheral side edges 127. The absorbent composite 126 may extend longitudinally the entire length of the garment from the front to the back waist edges, or it may extend less than the overall length of the absorbent garment as shown in FIG. 6. The absorbent composite 126 includes a substantially liquid permeable topsheet or liner 104, which presents a body-facing surface that is compliant, soft-feeling, and non-irritating to the wearer's skin. The absorbent composite 126 also includes a substantially liquid impermeable backsheet or barrier layer 102 and a retention portion 106 disposed or sandwiched between the topsheet and the backsheet, which are connected at least along their peripheral edges. The materials of which the topsheet, backsheet and retention portion may be made are the same materials discussed above for those components of the first embodiment of the absorbent garment of this invention.

The absorbent composite 126, and in particular the retention portion 106, can be made in a variety of different shapes, including the rectangular shape shown FIG. 6, and also hour-glass shapes. Preferably, the shape of the portion of the absorbent composite 126 which is attached to the back body panel 124 widens out laterally as it extends from the crotch region longitudinally towards its back terminal end region 129.

The topsheet and backsheet can be attached at their peripheral edges, or they may be attached across some or all of the entire surface area of absorbent composite 126. Their attachment can be by various attachment techniques as will be known by those of skill in the art, such as, e.g., with adhesive bonds, sonic bonds, thermal bonds, pinning, stitching, among others. Additional layers, including a surge layer (not shown), can be incorporated into absorbent composite 126.

For the absorbent garment 120 of this embodiment, the undesirable “diaper” look shown in FIG. 1b is avoided by inclusion of rear defining member 142, as shown in FIG. 6. Rear body defining member 142 is formed and attached to the garment in the same way as described above for the embodiment shown in FIG. 3. In this embodiment, as indicated in FIG. 6, the rear body defining member 142 is attached to the absorbent garment through the backsheet 102 and through some or all of the other layers of absorbent composite 126, as well as through any other additional garment-side layers such as the back body panel 124. The result is the improved form-fitted and contoured look of the absorbent garment shown in FIG. 2. In addition, the shape and contours of the rear portion of absorbent composite 126 can also be formed to improve the contoured look of the garment. In a particularly preferred embodiment, absorbent composite 126 has an hour-glass shape and elastic members (not shown) are attached along peripheral leg edges 137 and 139 and peripheral edges 127 of the absorbent composite 126, which enhance the desired contoured look achieved by attachment of the rear body defining member 142 to absorbent garment 120. If the absorbent garment includes containment flaps, those flaps must be positioned and attached as for the previously discussed embodiment to maintain the desired fitted and contoured look for the garment.

Although the present invention has been described with reference to preferred embodiments, those skilled in the art will recognize that changes may be made in form and detail without materially departing from the novel teachings, advantages, and scope of this invention. Accordingly, it is intended that the foregoing detailed description be regarded as illustrative rather than limiting and that it is the appended claims, including all equivalents thereof, which are intended to define the scope of the invention.

What is claimed is:

1. An absorbent garment comprising:
an absorbent composite having a length, opposite front and rear ends, a crotch region positioned lengthwise between the opposite ends, opposite front side regions, and opposite rear side regions, said absorbent composite comprising a backsheet, a topsheet and a retention portion disposed between the backsheet and topsheet;
a pair of front body panels connected to said opposite front side regions adjacent the front end of said absorbent composite;
a pair of rear body panels connected to said opposite rear side regions adjacent the rear end of said absorbent composite; and
an elasticized rear defining member attached through the backsheet and the retention portion of said absorbent composite along a portion of the length of the absorbent composite between its crotch region and its rear end and midway between its opposite rear side regions.

2. The absorbent garment of claim 1, wherein said elasticized rear defining member comprises elastic stitching.

3. The absorbent garment of claim 1, wherein the portion of said absorbent composite between its rear end and its crotch region forms a rear back region, and said elasticized rear defining member causes said rear back region to become shaped into two defined and generally rounded portions on opposite sides of said rear defining member.

4. The absorbent garment of claim 3, wherein the lateral width of said absorbent composite is wider in the rear back region than in the crotch region.

5. The absorbent garment of claim 4 further comprising a pair of elasticized flaps attached to the topsheet of said absorbent composite along opposite lateral sides of the crotch region of the absorbent composite.

6. An absorbent garment comprising:
an absorbent composite having a length, opposite front and rear regions with a crotch region positioned lengthwise between them, opposite front sides having side edges, and opposite rear sides having side edges, said absorbent composite comprising a backsheet, a topsheet and a retention portion disposed between the backsheet and topsheet;
fastening components connected to the side edges of said front and rear sides for attaching the front sides to the rear sides; and
a rear body defining member attached to the absorbent garment and extending along at least a portion of the length of the rear region of the absorbent composite midway between its opposite rear sides, said rear body defining member causing the rear region of said absorbent garment to become shaped into two defined and generally rounded portions on opposite sides of said rear defining member.

7. The absorbent garment of claim 6, wherein said rear body defining member comprises elastic stitching which is stitched through the backsheet and retention portion of the absorbent composite.

8. A pant-like absorbent garment, comprising:
an absorbent garment having a garment-side surface and a body-side surface, and defining a waist opening and first and second leg openings, said absorbent garment further defining a longitudinal axis, a lateral axis, front and back waist edges parallel to the lateral axis, opposite side edges extending between said front and back waist edges, a front region contiguous with the front waist edge, a back region contiguous with the back waist edge, and a crotch region which extends between and interconnects the front and back regions, said absorbent garment comprising:
a composite structure comprising a liquid permeable body-side topsheet, an liquid impermeable backsheet, and an absorbent retention assembly disposed between the body-side topsheet and the backsheet, said composite structure disposed in the crotch region and extending longitudinally into the front and back regions;
first and second front side panels in the front region extending to opposite side edges;
first and second back side panels in the back region extending to opposite side edges;
a fastening system for releasably securing the absorbent garment in a pant-like configuration, said fastening system comprising fastening components connected to the side edges of the front and back side panels;
elastomeric components disposed along one or more of the front and back waist edges, the leg openings, and the sides of the crotch region; and
a rear body defining member attached to said absorbent garment and extending along at least a portion of the length of the back region midway between its opposite back side panels, said rear body defining member causing the back region of the absorbent garment to become shaped into two defined and generally rounded portions on opposite lateral sides of said rear defining member.

9. The pant-like absorbent garment of claim 8, wherein said rear body defining member comprises elastomeric stitching which is stitched through the backsheet and absorbent retention assembly the composite structure.

10. An absorbent garment comprising:
longitudinally spaced first and second body panels each having a body-side surface, a terminal waist edge, and a terminal crotch edge, wherein said crotch edges of said first and second body panels are longitudinally spaced apart and define a gap therebetween and wherein said waist edges of said first and second body panels are longitudinally spaced apart;
an absorbent composite having a longitudinally extending length, a laterally extending width, and longitudinally opposed first and second end regions having respective first and second ends, said absorbent composite comprising a backsheet, a topsheet, and a retention portion disposed between said backsheet and topsheet, wherein said absorbent composite bridges said gap between said crotch edges of said first and second body panels and wherein said first end region of said absorbent composite overlaps and is connected to said body-side surface of said first body panel along a longitudinally extending center portion thereof and across an entirety of said width of said absorbent composite at said first end thereof, and wherein said second end region of said absorbent composite overlaps and is connected to said body-side surface of said second body panel along a longitudinally extending center portion thereof and across an entirety of said width of said absorbent composite at said second end thereof; and
an elasticized rear body defining member attached to said absorbent garment along at least a portion of the length of one of said body panels along the panel's longitudinal center line and being attached through the body panel and layers of the absorbent composite attached to the body panel, said rear body defining member causing the body panel and absorbent composite to which it is attached to become shaped into two defined and generally rounded portions of said absorbent garment on laterally opposite sides of said rear defining member.
